(19) United States
(12) Patent Application Publication
(10) Pub. No.: US 2019/0167548 A1
(43) Pub. Date: Jun. 6, 2019

(54) RESTRUCTURING OF BLEACHED HAIR BY AGENTS HAVING ALIPHATIC POLYOLS AND CYCLIC CARBONATES

(71) Applicant: Henkel AG & Co. KGaA, Duesseldorf (DE)

(72) Inventors: Konstantin Goutsis, Juechen (DE); Daniela Kessler-Becker, Leverkusen (DE); Jing Hodes, Hagen (DE)

(73) Assignee: Henkel AG & Co. KGaA, Duesseldorf (DE)

(21) Appl. No.: 16/201,208

(22) Filed: Nov. 27, 2018

(30) Foreign Application Priority Data
Dec. 1, 2017 (DE) 10 2017 221 630.1

(51) Int. Cl. A61K 8/34; A61Q 5/00; A61Q 5/08; A61K 8/49

(52) U.S. Cl. CPC A61K 8/345; A61Q 5/002; A61K 2800/884; A61K 8/4973; A61Q 5/08

(57) ABSTRACT

An agent (M) for the treatment of keratin fibers, in particular human hair, containing in a cosmetic carrier, based on the total weight of the agent (M),
(a) one or more aliphatic polyols in a total amount from about 1.0 to about 40.0% by weight and
(b) one or more cyclic carbonates in a total amount from about 1.0 to about 20.0% by weight, wherein the weight ratio of the total amount of the aliphatic polyols (a) contained in the agent (M) to the total amount of the cyclic carbonates (b) contained in the agent (M), that is, the weight ratio (a)/(b), is a value of from about 40 to about 1.

### CROSS-REFERENCE TO RELATED APPLICATION

This application claims priority to German Patent Application No. 10 2017 221 630.1, filed Dec. 1, 2017, which is incorporated herein by reference in its entirety.

### TECHNICAL FIELD

The present disclosure is in the field of cosmetics and relates to agents for the restructuring of damaged, in particular bleached, keratin fibers which contain aliphatic polyols and cyclic carbonates in certain quantity ranges and quantity proportions.

### BACKGROUND

Changing the color of keratin fibers, especially hair, represents an important area of modern cosmetics. Through this, the appearance of the hair can be adapted to both current fashion trends and the individual wishes of the individual person. The person skilled in the art knows different options for changing the color of the hair. The hair color can be changed temporarily through the use of direct dyes. Oxidative color changing agents are usually used when the consumer desires a long-lasting color result or a shade that is lighter than his original hair color.

Hydrogen peroxide is usually used as the oxidizing agent of choice in oxidative color changing agents. Hydrogen peroxide not only initiates the dye formation process, but also destroys the hair's own melanin pigments in an oxidative process. A simultaneous lightening of the hair is achieved as a result. The amount of hydrogen peroxide used is usually chosen as a function of the desired lightening effect. Oxidative coloring agents usually contain amounts between 3 and 12% by weight of hydrogen peroxide.

The pure lightening or bleaching of hair is often done by using oxidizing agents without the addition of oxidation dye precursors. For a medium bleaching effect, the use of hydrogen peroxide alone is sufficient as the oxidizing agent; for the achievement of a stronger bleaching effect, a mixture of hydrogen peroxide and peroxydisulfate salts is usually used.

However, damage to the hair inevitably occurs with every lightening, since not only the melanin pigments, but also the other structural components of the hair are oxidatively damaged. The melanin pigments are arranged in the form of granules in the cortex cells, that is, in the interior of the hair. With the oxidative destruction of the melanin granules, oxidizing agents such as hydrogen peroxide and/or persulfates not only damage the cuticle layer of the hair, but also oxidatively degrade other structural components located inside the hair fiber.

The most important building blocks of hair fibers are the amino acids that are linked in the protein molecules of the keratin fibers by amide bonds to macromolecules. Due to the influence of the oxidizing agents, apart from melanin degradation, chemical degradation reactions also take place on these polypeptide chains, for example, on the cystine bonds, on the amide and amino groups and on the hydrogen bonds and salt bonds of the peptide structure. These degradation reactions lead to strong changes in the original mechanical properties of the hair.

Hair keratins are proteins having an exceptionally high proportion (up to 10% by weight) of cystine. The cystine covalently links two units of the amino acid cysteine with each other via a disulfide structural unit, the units being located in different peptide strands. The cystines contained in the hair are essential for the mechanical stability of the hair.

When oxidative color changing agents are used, a part of the cystine bonds present in the keratin fiber is now oxidatively degraded. Here, the disulfide groups of cystine are oxidatively cleaved and converted into sulfonic acid units. In this way, the cystine linking different peptide strands is oxidized to two discrete, that is, no longer bonded, cysteic acid units.

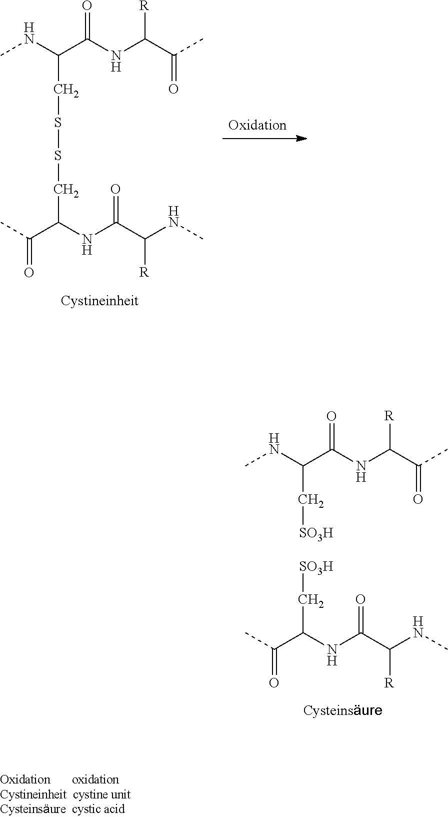

Due to these cleavage reactions, the number of linkage sites of the peptide strands with each other decreases, and the mechanical stability of the keratin fibers is massively impaired. The more often the oxidative color changing agents are applied and the higher the hydrogen peroxide content (either alone or in combination with other oxidizing agents such as persulfates) in the agents, the greater the loss of stability within the peptide backbone. This manifests itself in particular in a reduced resistance and reduced tear resistance of the keratin fibers. In addition, their extensibility, especially in their wet state, greatly increases. All these accompanying effects of oxidative color changing agents are extremely undesirable for the consumer.

There is therefore still a need for agents which can be applied before or after a bleaching agent and which can minimize or repair the hair damage caused by the bleaching.

Some users not only want a change in hair color, but also a simultaneous change in the hair shape, especially a smoothing of the curly hair or a waving of smooth hair. An increasingly popular method of hair straightening is smoothing with a hot straightening iron. However, the structure of the keratin-containing fiber changes during the heat treatment of the hair during the smoothing, so that the warmth or heat assisted smoothing process damages the hair to a greater or lesser extent.

The combination of a bleaching with a hair straightening is so far associated with such a strong hair damage that the user who performs both procedures in short time intervals one after the other must expect extremely great hair breakage, which can even amount to total loss of hair.

### BRIEF SUMMARY

This disclosure provides an agent (M) for the treatment of keratin fibers comprising, in a cosmetic carrier, based on the total weight of the agent (M), (a) one or more aliphatic polyols in a total amount from about 1.0 to about 40.0% by weight and (b) one or more cyclic carbonates in a total amount from about 1.0 to about 20.0% by weight, wherein the weight ratio of the total amount of the aliphatic polyols (a) included in the agent (M) to the total amount of the cyclic carbonates (b) included in the agent (M), (the weight ratio (a)/(b)), is a value of from about 40 to about 1.

This disclosure also provides a method of treating hair comprising the following steps in the order specified: (I) treating the hair with an agent (M), (II) letting the agent (M) act on the hair, (III) optionally rinsing off the agent (M) from the hair, and (IV) optionally drying the hair and (V) heat treatment of the hair.

This disclosure further provides a multi-component packaging unit (kit-of-parts), comprising at least two cosmetic agents (M) and (OX) assembled separately from each other, wherein the agent (M) is as described above and the agent (OX) is an oxidizing agent preparation comprising at least one oxidizing agent selected from the group of hydrogen peroxide, ammonium persulfate, sodium persulfate and potassium persulfate.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

One subject of the present disclosure is a method in which the damaged keratin fibers are treated with the agent mentioned above and subjected to a heat treatment afterwards.

A further subject of the present disclosure is a multi-component packaging unit which, assembled separately, contains at least two cosmetic preparations, wherein the first preparation is the above-described restructuring agent and the second preparation is an oxidizing agent preparation.

Finally, a further subject is the use of an agent which contains aliphatic polyols and cyclic carbonates in certain quantity ranges and quantity proportions for the restructuring or repair of oxidatively damaged hair.

Another object of the present disclosure was to provide agents with excellent restructuring effect. The hairs treated with these agents should be exemplified by a reduced cysteic acid content and possess improved mechanical stability, higher tear resistance and reduced extensibility. Here, the repairing or restructuring effect should be so high that the keratin fibers or hair can be bleached and smoothed in a temporally close sequence. In other words, application of the agent should reduce hair damage to a level acceptable to the user when performing combined bleaching and smoothing methods.

Surprisingly, it has now been found that the object described above can be achieved to the utmost satisfaction when the bleached/lightened hairs are treated with an agent that contains aliphatic polyols (a) and cyclic carbonates (b) in specific amounts and weight ratios.

A first subject of the present disclosure is therefore an agent (M) for the treatment of keratin fibers, in particular human hair, containing in a cosmetic carrier, based on the total weight of the agent (M),
(a) one or more aliphatic polyols in a total amount from about 1.0 to about 40.0% by weight and
(b) one or more cyclic carbonates in a total amount from about 1.0 to about 20.0% by weight, wherein the weight ratio of the total amount of the aliphatic polyols (a) contained in the agent (M) to the total amount of the cyclic carbonates (b) contained in the agent (M), that is, the weight ratio (a)/(b), is at a value of from about 40 to about 1.

Agent for the Treatment of Keratin Fibers

Keratin fibers, keratin-containing fibers or keratin fibers are understood to mean furs, wool, feathers and, in particular, human hair. The agents as contemplated herein are cosmetic agents which are applied to keratin fibers, in particular human hair, and which reduce hair damage already present during use.

When, in principle, the agents as contemplated herein can of course also be used on intact hairs, then it is of particular advantage to apply the agents to already damaged hair, since in this case, a reduction of hair damage can be detected particularly well.

In this context, damaged keratin fibers or hairs are understood to mean keratin fibers/hair which have been damaged by the usual environmental influences such as sunlight, repeated hair washing or abrasion of textiles. In particular, however, damaged hair for the purposes of the present disclosure is understood to mean oxidatively damaged hair, wherein the damage has been caused by (optionally repeated) application of an oxidative coloring agent, a lightening agent and/or a bleaching agent.

The agent as contemplated herein is thus, in particular, an agent (M) for the treatment of oxidatively damaged keratin fibers, in particular oxidatively damaged human hair, containing in a cosmetic carrier, based on the total weight of the agent (M),
(a) one or more aliphatic polyols in a total amount from about 1.0 to about 40.0% by weight and
(b) one or more cyclic carbonates in a total amount from about 1.0 to about 20.0% by weight, wherein the weight ratio of the total amount of the aliphatic polyols (a) contained in the agent (M) to the total amount of the cyclic carbonates (b) contained in the agent (M), that is, the weight ratio (a)/(b), is at a value of from about 40 to about 1.

In other words, in particular, it is an agent (M) for the treatment of bleached or lightened keratin fibers, in particular bleached or lightened human hair, contained in a cosmetic carrier, based on the total weight of the agent (M),
(a) one or more aliphatic polyols in a total amount from about 1.0 to about 40.0% by weight and
(b) one or more cyclic carbonates in a total amount from about 1.0 to about 20.0% by weight, wherein the weight ratio of the total amount of the aliphatic polyols (a) contained in the agent (M) to the total amount of the cyclic carbonates (b) contained in the agent (M), that is, the weight ratio (a)/(b), is at a value of from about 40 to about 1.

For the purposes of the present disclosure, the reduction of the damage to the keratin fibers is understood to mean the reduction of the structural damage to the protein scaffold responsible for the mechanical strength of the fiber, wherein the reduction of the structural damage may occur in the cortex, that is, in the interior of the fiber and/or also on the cuticle, that is, the outer cuticle layer of the keratin fibers.

The agents as contemplated herein contain the essential ingredients (a) and (b) each in a cosmetic carrier, preferably in a suitable aqueous, alcoholic or aqueous-alcoholic carrier. For the purpose of hair treatment, such carriers may be, for example, creams, emulsions, gels or surfactant-containing foaming solutions such as shampoos, foam aerosols, foam formulations or other preparations suitable for use on the hair. The agents as contemplated herein are, with particular preference, creams or emulsions.

Aliphatic Polyols (a)

As a first constituent of the present disclosure, the agents as contemplated herein contain one or more aliphatic polyols (a) in a total amount from about 1.0 to about 40.0% by weight. All data specified in % by weight are based on the total amount of the aliphatic polyols (a) present in the agent, which is set in relation to the total weight of the agent.

For the purposes of the present disclosure, aliphatic polyols are understood to mean aliphatic (that is, nonaromatic) di- or polyhydroxy compounds having from about 2 to about 10 carbon atoms, which are also referred to as polyalcohols or polyhydroxy compounds. The polyols carry at least two hydroxyl groups, but may also include 3, 4, 5 or 6 hydroxy groups.

Aliphatic polyols which are particularly suitable for reducing hair damage are compounds which carry from about 2 or about 3 hydroxyl groups. Particularly preferably, the aliphatic polyols have no further functional groups in addition to the hydroxy groups.

Particularly preferred is an agent (M) for the treatment of bleached or lightened keratin fibers, in particular bleached or lightened human hair, contained in a cosmetic carrier, based on the total weight of the agent (M),
(a) one or more aliphatic polyhydroxy compounds having from about 2 to about 10 carbon atoms in a total amount from about 1.0 to about 40.0% by weight and
(b) one or more cyclic carbonates in a total amount from about 1.0 to about 20.0% by weight, wherein the weight ratio of the total amount of the aliphatic polyols (a) contained in the agent (M) to the total amount of the cyclic carbonates (b) contained in the agent (M), that is, the weight ratio (a)/(b), is at a value of from about 40 to about 1.

Particularly suitable aliphatic polyols (a) are selected from the group of 1,2-propanediol, 1,2-ethanediol, 1,2,3-propanetriol, 1,3-propanediol, 1,2-butanediol, 1,3-butanediol and 1,4-butanediol, more preferably selected from 1,2-propanediol and 1,2-ethanediol.

1,2-propanediol is also referred to as 1,2-propylene glycol or 1,2-dihydroxypropane and has CAS numbers 57-55-6 [(RS)-1,2-dihydroxypropane], 4254-14-2 [(R)-1,2-dihydroxypropane] and 4254-15-3 [(S)-1,2-dihydroxypropane]. All stereoisomers are included in the present disclosure.

1,2-ethanediol is also referred to as ethylene glycol or 1,2-dihydroxyethane and has the CAS number 107-21-1.

1,2,3-propanetriol also bears the name glycerol and has the CAS number 56-81-5.

1,3-propanediol is alternatively referred to as trimethylene glycol or as 1,3-dihydroxypropane and has the CAS number 504-63-2.

1,2-butanediol is alternatively also called 1,2-butylene glycol and has the CAS numbers 584-03-2, 40348-66-1 (R) form, 73522-17-5 (S) form, and 26171-83-5 (RS) isomer mixture. All stereoisomers are encompassed by the present disclosure.

1,3-butanediol is alternatively also referred to as 1,3-butylene glycol and has the CAS numbers 107-88-0 (Racemate), 6290-03-5 [(R)-1,3-butanediol], 24621-61-2 [(S)-(+)-1,3-butanediol]. All stereoisomers are encompassed by the present disclosure.

Finally, 1,4-butanediol bears the alternative name tetramethylene glycol and has the CAS number 110-63-4.

Very particularly preferred aliphatic polyols (a) are selected from the group of 1,2-propanediol and 1,2-ethanediol, most preferably 1,2-propanediol.

In a particularly preferred embodiment, an agent as contemplated herein is therefore exemplified in that the agent (M) contains
(a) one or more aliphatic polyols (a) selected from the group of 1,2-propanediol, 1,2-ethanediol, 1,2,3-propanetriol, 1,3-propanediol, 1,2-butanediol, 1,3-butanediol and 1,4-butanediol, more preferably selected from 1,2-propanediol and 1,2-ethanediol.

The aliphatic polyol(s) (a) is/are contained in a total amount from about 1.0 to about 40.0% by weight in the agent as contemplated herein. At the same time, the ratio condition that must be met is that the weight ratio of the total amount of the aliphatic polyols (a) contained in the agent (M) to the total amount of the cyclic carbonates (b) contained in the agent (M), that is, the weight ratio (a)/(b), is at a value of from about 40 to about 1.

The use of the aliphatic polyols (a) in very specific quantity ranges has proved to be particularly advantageous for achieving the object of the present disclosure. When the agents contain one or more aliphatic polyols (a) in a total amount from about 5.0 to about 20.0% by weight, preferably from about 8.0 to about 18.0% by weight, more preferably from about 9.0 to about 16.0% by weight and most preferably from about 11.0 to about 16.0% by weight, all existing hair damage could be particularly effectively minimized, and the hair damage that was caused by a combined bleaching and smoothing application was quite especially low.

In a very particularly preferred embodiment, an agent as contemplated herein is therefore exemplified in that the agent (M), based on its total weight, contains
(a) one or more aliphatic polyols in a total amount from about 5.0 to about 20.0% by weight, preferably from about 8.0 to about 18.0% by weight, more preferably from about 9.0 to about 16.0% by weight, and most preferably from about 11.0 to about 16.0% by weight.

Cyclic Carbonates (b)

As a second constituent of the present disclosure, the agents as contemplated herein contain one or more cyclic carbonates (b) in a total amount from about 1.0 to about 20.0% by weight. All data specified in % by weight are based on the total amount of the cyclic carbonates (b) present in the agent, which is set in relation to the total weight of the agent.

For the purposes of the present disclosure, cyclic carbonates (b) are understood to mean the cyclic esters of carbonic acid and dihydroxy compounds.

Suitable cyclic carbonates (b) are, for example, the compounds of the general formula (I)

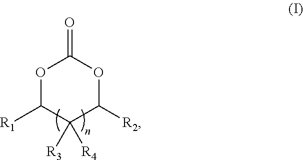

wherein
R1, R2 independently of one another stand for a hydrogen atom, a C1-C6 alkyl group, a hydroxy-C1-C6 alkyl group or a hydroxy group, and
R3, R4 independently of one another stand for a hydrogen atom, a C1-C6 alkyl group, a hydroxy-C1-C6 alkyl group or a hydroxy group, and
n stands for the numbers 0, 1 or 2.

The radicals R1 and R2 independently of one another preferably stand for a hydrogen atom, a methyl group, an ethyl group or a hydroxymethyl group.

The radicals R3 and R4 preferably stand for a hydrogen atom, a methyl group, an ethyl group or a hydroxymethyl group.

n preferably stands for the numbers 0 or 1, very particularly preferably n stands for the number 0. When n stands for the number 0, the compound of formula (I) contains no grouping —CR3R4-, but rather there is a direct bond between the two carbon atoms which carry the radicals R1 and R2.

Particularly suitable cyclic carbonates (b) are, for example, the compounds of the general formula (Ia)

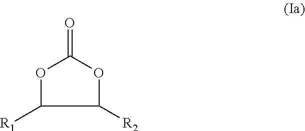

wherein
R1, R2 independently of one another stand for a hydrogen atom, a C1-C6 alkyl group or a hydroxy-C1-C6 alkyl group.

The cyclic carbonates that are selected from the group of ethylene carbonate, propylene carbonate, butylene carbonate and glycerol carbonate have proven particularly suitable for minimizing hair damage caused by oxidative processes.

Ethylene carbonate is also referred to as 1,3-dioxolan-2-one. Ethylene carbonate corresponds to the compound of the formula (I) in which R1 and R2 stand for hydrogen and n stands for the number 0. Ethylene carbonate has the CAS number 96-49-1.

Propylene carbonate is alternatively referred to as 4-methyl-1,3-dioxolan-2-one. Propylene carbonate corresponds to the compound of the formula (I), in which R1 stands for a methyl group, R2 stands for hydrogen and n stands for the number 0. Propylene carbonate has the CAS numbers 108-32-7 [(RS)-4-methyl-1,3-dioxolan-2-one], 51260-39-0 [(S)-4-methyl-1,3-dioxolan-2-one] and 16606-55-6 [(R)-4-methyl-1,3-dioxolan-2-one]. All of the aforementioned stereoisomers are encompassed by the present disclosure.

Butylene carbonate is understood as contemplated herein to mean the 1,2-butylene carbonate, which is alternatively also referred to as 4-ethyl-1,3-dioxolan-2-one and which has the CAS number 4437-85-8. Butylene carbonate corresponds to the compound of the formula (I), in which R1 stands for an ethyl group, R2 stands for a hydrogen atom and n stands for the number 0.

Glycerol carbonate is alternatively referred to as 4-hydroxymethyl-1,3-dioxolan-2-one and has the CAS number 931-40-8. Glycerol carbonate corresponds to the compound of the formula (I), in which R1 stands for a hydroxymethyl group, R2 stands for a hydrogen atom and n stands for the number 0.

In a particularly preferred embodiment, an agent as contemplated herein is therefore exemplified in that the agent (M) contains
(b) one or more cyclic carbonates selected from the group of ethylene carbonate, propylene carbonate, butylene carbonate and glycerol carbonate, more preferably selected from ethylene carbonate and propylene carbonate.

The cyclic carbonate(s) (b) is/are contained in a total amount from about 1.0 to about 20.0% by weight in the agent as contemplated herein. At the same time, the ratio condition that must be met is that the weight ratio of the total amount of the aliphatic polyols (a) contained in the agent (M) to the total amount of the cyclic carbonates (b) contained in the agent (M), that is, the weight ratio (a)/(b), is at a value of from about 40 to about 1.

The use of the cyclic carbonates (b) in very specific quantity ranges has proved to be particularly advantageous for achieving the object of the present disclosure. When the agents contain one or more cyclic carbonates (b) in a total amount from about 2.0 to about 15.0% by weight, preferably from about 3.0 to about 14.0% by weight, more preferably from about 4.0 to about 11.0% by weight, and most preferably from about 4.0 to about 9.0% by weight, all existing hair damage could be minimized particularly effectively, and the hair damage was very low in a combined bleaching and smoothing application.

In a very particularly preferred embodiment, an agent as contemplated herein is therefore exemplified in that the agent (M), based on its total weight, contains (b) one or more cyclic carbonates in a total amount from about 2.0 to about 15.0% by weight, preferably from about 3.0 to about 14.0% by weight, more preferably from about 4.0 to about 11.0% by weight, and most preferably from about 4.0 to about 9.0% by weight.

Quantity Proportion (a)/(b)

Furthermore, the agents contain the aliphatic polyols (a) and the cyclic carbonates (b) in very specific quantity proportions to each other. Here, the weight ratio of the total amount of the aliphatic polyols (a) contained in the agent (M) to the total amount of the cyclic carbonates (b) contained in the agent (M), that is, the weight ratio (a)/(b), is at a value of from about 40 to about 1.

Typically, the weight ratio (a)/(b) is at a value which is at least about 1, which means that the total amount of aliphatic polyols (a) used in the agent must be at least as large as the total amount of the cyclic carbonates (b) contained in the agent.

Example: an agent as contemplated herein contains
(a) 15.0% by weight of 1,2-propanediol and
(b) 5.0% by weight of ethylene carbonate
The weight ratio (a)/(b) is 15.0% by weight/5.0% by weight=3.

Example: 100 of the agent as contemplated herein contain
(a) 5.0 g of 1,2-propanediol and 10.0 g of 1,2-ethanediol and
(b) 2.5 g of ethylene carbonate and 2.5 g of propylene carbonate
The weight ratio (a)/(b) is [5.0 g+10.0 g]/[2.5 g+2.5 g]=3

It has been found to be particularly preferred when the aliphatic polyols (a) are used in excess in comparison to the cyclic carbonates (b), that is, when the weight ratio of the total amount of the aliphatic polyols (a) contained in the agent to the cyclic carbonates (b) contained in the agent is at a value of greater than about 1.

In this context, it has proved to be particularly favorable and therefore preferred as contemplated herein, when the weight ratio of the total amount of the aliphatic polyols (a) contained in the agent (M) to the total amount of cyclic carbonates (b) contained in the agent (M), that is, the weight ratio (a)/(b), is at a value of from about 10 to about 1, preferably from about 5 to about 1, more preferably from about 4 to about 1 and most preferably from about 3 to about 2.

In a very particularly preferred embodiment, an agent as contemplated herein is therefore exemplified in that the weight ratio of the total amount of the aliphatic polyols (a) contained in the agent (a) to the total amount of the cyclic carbonates (b) contained in the agent (M), that is, the weight ratio (a)/(b), is at a value of from about 10 to about 1, preferably from about 5 to about 1, more preferably from about 4 to about 1 and most preferably from about 3 to about 2.

It is thus preferred that an agent (M) for the treatment of keratin fibers, in particular human hair, containing in a cosmetic carrier, based on the total weight of the agent (M),
(a) 1,2-propanediol and/or ethylene glycol in a total amount from about 1.0 to about 40.0% by weight and
(b) ethylene carbonate and/or propylene carbonate in a total amount from about 1.0 to about 20.0% by weight,
wherein the weight ratio of the total amount of the aliphatic polyols (a) contained in the agent (M) to the total amount of the cyclic carbonates (b) contained in the agent (M), that is, the weight ratio (a)/(b), is at a value of from about 40 to about 1.

It is thus preferred that an agent (M) for the treatment of keratin fibers, in particular human hair, containing in a cosmetic carrier, based on the total weight of the agent (M),
(a) 1,2-propanediol and/or ethylene glycol in a total amount from about 5.0 to about 20.0% by weight and
(b) ethylene carbonate and/or propylene carbonate in a total amount from about 2.0 to about 15.0% by weight,
wherein the weight ratio of the total amount of the aliphatic polyols (a) contained in the agent (M) to the total amount of the cyclic carbonates (b) contained in the agent (M), that is, the weight ratio (a)/(b), is at a value of from about 10 to about 1.

It is thus preferred that an agent (M) for the treatment of keratin fibers, in particular human hair, containing in a cosmetic carrier, based on the total weight of the agent (M),
(a) 1,2-propanediol and/or ethylene glycol in a total amount from about 8.0 to about 18.0% by weight and
(b) ethylene carbonate and/or propylene carbonate in a total amount from about 3.0 to about 14.0% by weight,
wherein the weight ratio of the total amount of the aliphatic polyols (a) contained in the agent (M) to the total amount of the cyclic carbonates (b) contained in the agent (M), that is, the weight ratio (a)/(b), is at a value of from about 5 to about 1.

It is thus preferred that an agent (M) for the treatment of keratin fibers, in particular human hair, containing in a cosmetic carrier, based on the total weight of the agent (M),
(a) 1,2-propanediol and/or ethylene glycol in a total amount from about 9.0 to about 16.0% by weight and
(b) ethylene carbonate and/or propylene carbonate in a total amount from about 4.0 to about 11.0% by weight,
wherein the weight ratio of the total amount of the aliphatic polyols (a) contained in the agent (M) to the total amount of the cyclic carbonates (b) contained in the agent (M), that is, the weight ratio (a)/(b), is at a value of from about 4 to about 1.

It is thus preferred that an agent (M) for the treatment of keratin fibers, in particular human hair, containing in a cosmetic carrier, based on the total weight of the agent (M),
(a) 1,2-propanediol and/or ethylene glycol in a total amount from about 11.0 to about 16.0% by weight and
(b) ethylene carbonate and/or propylene carbonate in a total amount from about 4.0 to about 9.0% by weight,
wherein the weight ratio of the total amount of the aliphatic polyols (a) contained in the agent (M) to the total amount of the cyclic carbonates (b) contained in the agent (M), that is, the weight ratio (a)/(b), is at a value of from about 3 to about 2.

pH Value

The agent as contemplated herein is preferably provided in the form of an aqueous or hydrous composition. Their pH value may be, for example, in the range of from about 2 to about 10. The aqueous or hydrous agents (M) preferably have a pH value of from about 5 to about 7. For the purposes of the present disclosure, the pH values are pH values measured at a temperature of about 22° C.

The alkalizing agents which can be used as contemplated herein for adjusting the preferred pH value can be selected from the group of ammonia, alkanolamines, basic amino acids and inorganic alkalizing agents such as (earth) alkali metal hydroxides, (earth) alkali metal metasilicates, (earth) alkaline metal phosphates and (earth) alkali metal hydrogen phosphates.

Suitable acidifying agents are, for example, mineral acids such as hydrochloric acid, sulfuric acid or phosphoric acid or else organic acids which can be selected, for example, from the group of citric acid, lactic acid, malic acid, succinic acid, maleic acid and/or benzoic acid.

Other Ingredients

The agents (M) as contemplated herein and described above may further contain additional active ingredients, auxiliaries and additives in order to adjust further desired properties of the agents.

The agents as contemplated herein are preferably provided as a liquid preparation and, if appropriate, an additional surface-active substance is additionally added to the agents, wherein such surface-active substances are referred to as surfactants or as emulsifiers, depending on the field of application: They are preferably selected from anionic, zwitterionic, amphoteric and nonionic surfactants and emulsifiers.

Agents suitable as contemplated herein are exemplified in that the agent additionally contains at least one anionic surfactant. Preferred anionic surfactants are fatty acids, alkyl sulfates, alkyl ether sulfates and ether carboxylic acids having from about 10 to about 20 carbon atoms in the alkyl group and up to about 16 glycol ether groups in the molecule.

Agents suitable as contemplated herein are exemplified in that the agent additionally contains at least one zwitterionic surfactant. Preferred zwitterionic surfactants are betaines, N-alkyl-N,N-dimethylammonium glycinates, N-acylaminopropyl-N,N-dimethylammonium glycinates, and 2-alkyl-3-carboxymethyl-3-hydroxyethylimidazolines. A preferred zwitterionic surfactant is known by the INCI name Cocamidopropyl Betaine.

Agents suitable as contemplated herein are exemplified in that the agent additionally contains at least one amphoteric surfactant. Preferred amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids. Particularly preferred amphoteric surfactants are N-cocoalkylaminopropionate, cocoacylaminoethylaminopropionate and C12-C18 acylsarcosine.

Furthermore, it has proved to be advantageous when the agents contain other, nonionic surface-active substances. Preferred nonionic surfactants have proved to be alkyl polyglycosides and alkylene oxide addition products of fatty alcohols and fatty acids with in each case from about 2 to about 30 moles of ethylene oxide per mole of fatty alcohol or fatty acid. Preparations having excellent properties are also obtained when they contain fatty acid esters of ethoxylated glycerol as nonionic surfactants.

The nonionic, zwitterionic or amphoteric surfactants are used in proportions from about 0.1 to about 45% by weight, preferably from about 1 to about 30% by weight and very particularly preferably from about 1 to about 15% by weight, based on the total amount of the agents.

The agents as contemplated herein may also contain at least one thickening agent. There are no fundamental restrictions with regard to these thickeners. Both organic and purely inorganic thickening agents can be used.

Suitable thickening agents are anionic, synthetic polymers, cationic synthetic polymers, naturally occurring thickening agents such as nonionic guar gums, scleroglucan gums or xanthan gums, gum arabic, ghatti gum, karaya gum, gum tragacanth, carrageenan gum, agar agar, locust bean flour, pectins, alginates, starch fractions and derivatives such as amylose, amylopectin and dextrins, and cellulose derivatives such as methylcellulose, carboxyalkylcelluloses and hydroxyalkylcelluloses, nonionic fully synthetic polymers such as polyvinyl alcohol or polyvinylpyrrolidinone; and inorganic thickening agents, in particular phyllosilicates such as bentonite, especially smectites, such as montmorillonite or hectorite.

Furthermore, the agents as contemplated herein may contain other active ingredients, auxiliaries and additives, for example nonionic polymers such as vinylpyrrolidinone/vinyl acrylate copolymers, polyvinylpyrrolidinone, vinylpyrrolidinone/vinyl acetate copolymers, polyethylene glycols and polysiloxanes; additional silicones, such as volatile or nonvolatile, straight-chain, branched or cyclic, crosslinked or uncrosslinked polyalkylsiloxanes (such as dimethicones or cyclomethicones), polyarylsiloxanes and/or polyalkylarylsiloxanes, in particular polysiloxanes with organofunctional groups, such as substituted or unsubstituted amines (amodimethicones), carboxyl, alkoxy and/or hydroxyl groups (dimethicone copolyols), linear polysiloxane (A)—polyoxyalkylene (B) block copolymers, grafted silicone polymers; cationic polymers such as quatemized cellulose ethers, polysiloxanes with quatemary groups, dimethyldiallylammonium chloride polymers, acrylamide-dimethyldiallyl-ammonium chloride copolymers, with diethyl sulfate quatemized dimethylaminoethylmethacrylate-vinylpyrrolidinone copolymers, vinylpyrrolidinone-imidazolinium methochloride copolymers, and quatemized polyvinyl alcohol; zwitterionic and amphoteric polymers; anionic polymers such as polyacrylic acids or crosslinked polyacrylic acids; structurants such as glucose, maleic acid and lactic acid, hair conditioning compounds such as phospholipids, for example, lecithin and cephalins; perfume oils, dimethylisosorbide and cyclodextrins; dyes for staining the agent; anti-dandruff ingredients such as piroctone olamine, zinc omadine and climbazole; amino acids and oligopeptides; protein hydrolysates based on animal and/or plant, and in the form of their fatty acid condensation products or optionally anionically or cationically modified derivatives; vegetable oils; sunscreens and UV blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetration substances such as propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescing agents such as ethylene glycol mono- and distearate and PEG-3-distearate; pigments and propellants such as propane-butane mixtures, N₂O, dimethyl ether, CO₂ and air.

The selection of these further substances will be made by the person skilled in the art according to the desired properties of the agents. With regard to further optional components and the amounts of these components used, reference is expressly made to the relevant manuals known to the person skilled in the art. The additional active ingredients and auxiliaries are preferably used in the agents as contemplated herein in amounts of from about 0.0001 to about 25% by weight, in particular from about 0.0005 to about 15% by weight, based on the total weight of the agents.

Method

The agents (M) as contemplated herein have a particularly good activity when used in a method in which the agents (M) are first applied to the hair and the hair treated with the agent is subsequently subjected to a heat treatment.

A second subject of the present disclosure is therefore a method of treating hair comprising the following steps in the order given:
(I) treating the hair with an agent (M),
(II) letting the agent (M) act on the hair,
(III) optionally rinsing off the agent (M) from the hair,
(IV) optionally drying the hair and
(V) heat treatment of the hair,
wherein the agent (M) is an agent as disclosed in detail in the description of the first subject of the present disclosure.

A second subject of the present disclosure is thus a method of treating hair comprising the following steps in the order specified:
(I) treating the hair with an agent (M),
(II) letting the agent (M) act on the hair,
(III) optionally rinsing off the agent (M) from the hair,
(IV) optionally drying the hair and
(V) heat treatment of the hair,
wherein the agent (M), based on its total weight, contains
(a) one or more aliphatic polyols in a total amount from about 1.0 to about 40.0% by weight and
(b) contains one or more cyclic carbonates in a total amount from about 1.0 to about 20.0% by weight, and wherein the weight ratio of the total amount of the aliphatic polyols (a) contained in the agent (M) to the total amount of the cyclic carbonates (b) contained in the agent (M), that is, the weight ratio (a)/(b), is at a value of from about 40 to about 1.

Characteristic of the method as contemplated herein is the use of the agent (M) as contemplated herein on the hair (that is, the steps (I) and (II) of the method) and the heat treatment of the hair (that is, step (V)).

Since the agent (M) is particularly well suited to repair or restructure oxidatively damaged hair, the agent (M) is preferably applied to bleached or lightened hair.

A very particularly preferred embodiment is therefore a method comprising
(I) treating bleached and/or lightened hair with the agent (M).

The bleaching or lightening of the hair in this case can be effected, for example, by applying an oxidizing agent preparation (OX), wherein this oxidizing agent preparation (OX) contains at least one oxidizing agent selected from the group of hydrogen peroxide, ammonium persulfate, sodium persulfate and potassium persulfate.

Preferred in this context is a method of treating hair comprising the following steps in the order specified:
(I′) treating the hair with an oxidizing agent preparation (OX) which contains at least one oxidizing agent selected from the group of hydrogen peroxide, ammonium persulfate, sodium persulfate and potassium persulfate,
(II′) letting the oxidizing agent preparation (OX) act on the hair,
(III′) rinsing the oxidizing agent preparation (OX),
(I) treating the hair with an agent (M),
(II) letting the agent (M) act on the hair,
(III) optionally rinsing off the agent (M) from the hair,
(IV) optionally drying the hair and
(V) heat treatment of the hair,
wherein the agent (M) is an agent as disclosed in detail in the description of the first subject of the present disclosure.

Without being bound to this theory, it is assumed that the combination of active substances contained in the agent (M), that is, the aliphatic polyols (a) and the cyclic carbonates (b), can incur covalent bonds with certain structural components of the hair when exposed to heat. In particular, the structural components which are formed after oxidative treatment of the hair appear to react particularly well with the aliphatic polyols (a) and cyclic carbonates (b). In this way, the structural damage of the hair caused by oxidative processes can be repaired or restructured.

The steps (I′), (II′) and (III′) can be carried out, for example, as part of a lightening or bleaching process. Since the steps (I′), (II′) and (III′) belong to one application, they are usually carried out in temporal close succession. After completion of step (III′), the hair is then lightened or bleached or color-changed through oxidation, but also damaged.

The treatment of the hair lightened or bleached in this manner with the agent (M) can now be done directly after bleaching. In the context of this embodiment, the user can treat the still moist hair with the agent (M) after step (III′).

Likewise, however, there may also be a certain period of time, for example a few hours or several days, between the completion of step (III′) and the beginning of step (I).

In particular, when there is a time interval between steps (III′) and (I), the hairs are preferably dried after completion of step (III′). In this case, the agent (M) in step (I) is preferably applied to the dry hair.

The treatment of the hair with the agent (M) in step (I) can, for example, be done by applying the agent (M) with a paintbrush, through a nozzle or with a brush. The agent (M) can also be sprayed on the hair in the form of a spray application, or be prepared as a cream or gel and massaged by hand into the hair.

After application, the agent (M) is allowed to act on the hair. For this purpose, the agent (M) is preferably left for a period of from about 2 to about 45 minutes, preferably from about 2 to about 30 minutes, more preferably from about 2 to about 20 minutes and particularly preferably from about 2 to about 10 minutes (step (II)).

After the action, the agent (M) can be rinsed off the hair in one embodiment (step (III)). Within the scope of a further embodiment, the agent (M) as contemplated herein is designed as a “leave-on” product and left on the hair.

The hairs which are still applied with the agent (M) are optionally dried (step IV) and then subjected to the heat treatment (step V). The preparation in the form of a “leave-on” product is preferred, that is, particularly preferably the hair is not rinsed off after the action of the agent (M).

In step (IV) of the method, the hairs can optionally be dried. The hair is preferably dried, since the subsequent heat treatment (step (V)), especially when these higher temperatures act on the hair, may be associated with increased hair damage on wet hair.

A dry hair fiber is present when the residual water adhering to the hair has evaporated to the extent that the hairs fall individually. Preferably, in a dry keratin-containing fiber, either the moisture content of the fiber is substantially in equilibrium with the humidity of the air, or the fiber absorbs moisture from the ambient air. Such a dry fiber is preferably achieved by drying the wet fiber with hot air using a hair dryer. The drying in step (IV) is preferably carried out when, in step (V) of the method as contemplated herein, a heat treatment takes place in which the surface of the keratin-containing fibers takes place directly with a tempered surface of a shaping aid (for example, straightening iron or waving iron) during a forming step.

In a further embodiment, a method comprising the following steps in the order specified is therefore very particularly preferred
(I) treating the hair with an agent (M),
(II) letting the agent (M) act on the hair for a period of from about 2 to about 45 minutes, preferably from about 2 to about 30 minutes, more preferably from about 2 to about 20 minutes and most preferably from about 2 to about 10 minutes,
(III) no rinsing off of the agent (M) from the hair,
(IV) drying the hair and
(V) heat treatment of the hair.

In other words, very particularly preferred is a method comprising the following steps in the order specified
(I) treating the hair with an agent (M),
(II) letting the agent (M) act on the hair for a period of from about 2 to about 45 minutes, preferably from about 2 to about 30 minutes, more preferably from about 2 to about 20 minutes and most preferably from about 2 to about 10 minutes,
(IV) drying the hair and
(V) heat treatment of the hair.

It is particularly preferred when steps (I) to (V) take place in direct temporal sequence one after the other. In other words, the best results have been obtained when, between the treatment of the keratin fibers with the agent (M) in step (I) and the heat treatment in step (V), there is a time interval of a maximum of about 24 hours, preferably of a maximum of about 12 hours, more preferably of a maximum of about 6 hours and most preferably of a maximum of about 3 hours.

The heat treatment in step (V) can be carried out in principle with any heat source such as, for example, a hair dryer. However, it has proved particularly advantageous to use a straightening iron or a waving iron as the heat source. In this way, the combination of a bleaching or lightening method with a hair deformation is possible without the user harming the hair excessively.

In a further very particularly preferred embodiment, a method as contemplated herein is exemplified in that the hair in step (V) is subjected to a heat treatment at a temperature from about 50° C. to about 350° C., preferably from about 80° C. to about 280° C., more preferably about 100° C. to about 260° C., even more preferably about 140° C. to about 250° C. and most preferably from about 170° C. to about 240° C.

In a particularly preferred embodiment, in step (V), the fibers are subjected to a heat treatment with simultaneous deformation of the fiber at a temperature from about 50° C. to about 350° C., preferably about 80° C. to about 280° C., more preferably about 100° C. to about 250° C., even more preferably about 140° C. to about 250° C. and most preferably from about 170° C. to about 240° C. Simultaneous deformation means that the fiber is brought or held during the heat treatment in a shape, for example, by mechanical smoothing or fixing of the shape by hair roller or papillotene.

In a further very particularly preferred embodiment, a method as contemplated herein is exemplified in that the hair in step (V) is subjected to a heat treatment with simultaneous deformation of the fiber at a temperature from about 50° C. to about 350° C., preferably from about 80° C. to about 280° C., more preferably about 100° C. to about 260° C., even more preferably about 140° C. to about 250° C. and most preferably from about 170° C. to about 240° C.

In a specific embodiment, in the context of a hair straightening, the fibers in step (V) are subjected to a heat treatment with mechanical smoothing of the fiber at a temperature of from about 50° C. to about 350° C., preferably from about 80° C. to about 280° C., more preferably from about 100° C. to about 260° C., even more preferably from about 140° C. to about 250° C. and most preferably from about 170° C. to about 240° C. The heat treatment can be done with hot air. In this case, the fiber is heated during the combing exactly at the point where the mechanical smoothing takes place. Moreover, it is particularly preferred that the heat treatment in the manner of smoothing with the aid of a so-called straightening iron with appropriately tempered plates, in particular metal or ceramic plates, takes place by pressing the plate onto the fiber to be smoothed and moving the plate pressed on the fiber along the fiber. The plates may optionally be coated with heat resistant substances. Particularly preferably, the keratin-containing fiber to be smoothed is pressed between two appropriately tempered plates and both plates are simultaneously moved along the longest spatial extent of the fiber. It is again preferred that both plates are connected to each other, so that both plates can be moved uniformly along the fiber. If the heat treatment is performed on living hair, the fiber is attached to one end (hair root). The plates in this case are preferably moved evenly away from the hair root along the entire fiber. This movement causes a mechanical smoothing of the fiber. A corresponding device for heat treatment, for example, is the device “Ceramic Flat-Master” (sold by: Efalock, Germany) or Schwarzkopf Professional: Model no: IP30A.

In a further embodiment, in the context of a hair waving, the fibers in step (V) are subjected to a heat treatment with mechanical curling of the fiber at a temperature from about 50° C. to about 350° C., preferably from about 80° C. to about 280° C., more preferably from about 100° C. to about 260° C., even more preferably from about 140° C. to about 250° C. and most preferably from about 170° C. to about 240° C. The heat treatment can be done with a hot air blower. In this case, either the fiber is wound around a corresponding hot air curling iron and heated exactly where the curling occurs, or the fibers are wound on curlers as a deformation aid and heated with a hair dryer. Another option for heat-assisted waving takes place in which the fibers wound around a rod with a correspondingly heated surface without using a hot air blower and heated exactly at the point where the curling occurs. A suitable device for heat treatment is, for example, the device from Schwarzkopf Professional, model no.: CPL Type F37.

In a further very particularly preferred embodiment, a method as contemplated herein is exemplified in that the heat source for the heat treatment in step (V) is a straightening iron or waving iron.

Concerning the further preferred embodiments of the method as contemplated herein, mutatis mutandis applies to the agent as contemplated herein (M).

Multi-Component Packaging Unit (Kit-of-Parts)

The agents and methods described above are used to repair or restructure damaged keratin fibers, in particular human hair. In particular, oxidative damage to the keratin fibers or hair can be optimally reduced by the use of the agents (M) as contemplated herein.

Therefore, it is particularly advantageous and convenient for the user when he is provided with a multi-component packaging unit (kit-of-parts) which comprises an agent for bleaching or lightening the hair and a further agent for reducing the hair damage caused by such treatment.

A further subject of the present disclosure is therefore a multi-component packaging unit (kit-of-parts) comprising at least two cosmetic agents (M) and (OX) assembled separately from each other, wherein
the agent (M) is an agent as was disclosed in detail in the description of the first subject of the present disclosure and
the agent (OX) is an oxidizing agent preparation containing at least one oxidizing agent selected from the group of hydrogen peroxide, ammonium persulfate, sodium persulfate and potassium persulfate.

The agents (M) and (OX) of the kit-of-parts are preferably applied successively on the keratin fibers, that is, first, the oxidizing agent preparation (OX) is applied, allowed to act and rinsed off again, and then afterwards the restructuring agent (M) is applied to the keratin fibers, allowed to act and rinsed off again.

As described above, drying of the keratin fibers is then particularly preferred, followed by heat treatment.

The oxidizing agent preparation (OX) contains at least one oxidizing agent selected from the group of hydrogen peroxide, ammonium persulfate, sodium persulfate and potassium persulfate.

Hydrogen peroxide is preferably used as the aqueous solution in the oxidizing agent preparation (OX). The concentration of a hydrogen peroxide solution in the oxidizing agent preparation (OX) is determined, on the one hand, by the legal requirements and, on the other hand, by the desired effect; preferably from about 1 to about 12% by weight solutions in water are used.

Oxidizing agent preparation (OX) which is preferred as contemplated herein is exemplified in that, based on the total weight of the oxidizing agent preparation (OX), it contains from about 1.5 to about 12.0% by weight, preferably from about 3.0 to about 11.0% by weight, more preferably from about 4.5 to about 10.0% by weight and particularly preferably from about 5.5 to about 9.5% by weight of hydrogen peroxide.

Potassium peroxodisulfate is alternatively referred to as potassium persulfate and has the molecular formula K₂S₂O₈.

Ammonium peroxodisulfate is alternatively referred to as ammonium persulfate and has the molecular formula (NH₄)₂S₂O₈.

Sodium peroxodisulfate is alternatively referred to as sodium persulfate and has the molecular formula Na₂S₂O₈.

Oxidizing agent preparation (OX) which is preferred as contemplated herein is exemplified in that, based on the total weight of the oxidizing agent preparation (OX), it contains one or more persulfates, in a total amount from about 5.0 to about 80.0% by weight, preferably from about 10.0 to about 50.0% by weight.

In a further embodiment, very particular preference is given to a multi-component packaging unit (kit-of-parts) comprising at least three cosmetic agents (M) and (OX) assembled separately from each other, wherein
the agent (M) is an agent as was disclosed in detail in the description of the first subject of the present disclosure and
the agent (OX1) is a first oxidizing agent preparation that contains hydrogen peroxide and
the agent (OX2) is a second oxidizing agent preparation that contains at least one oxidizing agent from the group of hydrogen peroxide, ammonium persulfate, sodium persulfate and potassium persulfate.

The oxidizing agent preparations (OX) (or the oxidizing agent preparations (O×1) and (O×2)) usually contain the oxidizing agent(s) in a cosmetic carrier, which is preferably aqueous, alcoholic or aqueous-alcoholic. For the purpose of hair treatment, such carriers may be, for example, creams, emulsions, gels or surfactant-containing foaming solutions such as shampoos, foam aerosols or other preparations suitable for use on the hair.

Regarding the further preferred embodiments of the multi-component packaging unit (kit-of-parts) as contemplated herein, mutatis mutandis applies to the agent (M) as contemplated herein.

Use

A further subject of the present disclosure is the use of an agent (M), as was disclosed in detail in the description of the first subject of the present disclosure, for the restructuring or repair of oxidatively damaged hair.

For the purposes of the present disclosure, the restructuring or repair of oxidatively damaged hair is understood to mean the reduction of the structural damage of the keratin fibers, which manifests itself in the increase of the tear resistance of the keratin fibers and/or the improvement of the tensile strength of the keratin fibers and/or the improvement of the combability of the (dry and/or wet) keratin fibers and/or the increase of the keratin melting point (measured by DSC or HP-DSC).

Concerning the further preferred embodiments of the method as contemplated herein, mutatis mutandis applies to the agent as contemplated herein (M).

### Examples

### 1. Formulations

The following formulations were prepared (all amounts in % by weight):

| | E1 (agent (M), | E2 (agent (M), | |
|---|---|---|---|
| | as contem- | as contem- | V |
| | plated | plated | (Com- |
| Agent (M) | herein) | herein) | parison) |
| 1,2-propanediol (a) | 15.0 | 10.0 | — |
| Ethylene carbonate (b) | 5.0 | — | — |
| Propylene carbonate (b) | — | 10.0 | — |
| Water (dist.) | ad 100 | ad 100 | ad 100 |
| Weight ratio (a)/(b) | 3 | 1 | — |

2.1. Test Method HP-DSC (High Pressure Differential Scanning Calorimetry)

Thermoanalytical examinations are particularly suitable for the characterization of two-phase systems, which include human hair as fiber keratins with their crystalline α-helix content and amorphous matrix content. On the one hand, glass transitions and aging behavior of the amorphous matrix can be examined, on the other hand, the melting behavior of the crystalline, helical phase provides important information about the hair structure.

For example, DSC (Differential Scanning Calorimetry) or HP-DSC (High Pressure Differential Scanning Calorimetry) methods are used as thermoanalytical examinations. In the HP-DSC method, DSC measurements are performed on keratins which are enclosed with water in commercially available pressure-tight measuring capsules. A high water vapor pressure develops in the keratin-water system when heated above 100° C. in the encapsulated steel crucibles, from which the HP-DSC analysis is derived. The major difference between the HP-DSC thermograms of human hair compared to normal DSC thermograms is that the endothermic peaks, which reflect the transformation point and transformation enthalpy, are shifted here to lower temperatures by about 90° C. This is because, after water diffuses into the hair fiber, weakening and cleavage of hydrogen bonds and salt bonds reduces the protein stability and thus lowers the “bonding temperature” of the keratins. If only hydrogen bonds and salt bonds are dissolved by the supercontracting agent, such as water, the thermal effect is reversible (supercontraction). However, the process becomes irreversible as soon as covalent bonds such as disulfide bonds are cleaved. This occurs when human hair fibers are heated to above 150° C. in pressure-resistant capsules with water. The irreversible transformation, interpreted as transition of the α-helical regions in the proteins to a disordered state, results in endothermic peaks, wherein the peak location reflects the transformation or denaturation point and the peak area reflects the transformation or denaturation enthalpy.

Using differential scanning calorimetry (DSC), structural and chemical states and changes can be detected in fiber keratins, and in human hair in particular. Under well-defined experimental conditions, the calorimetric detectable processes based on thermograms can be recorded in human hair and they can be used with respect to the peak positions, structures and areas as an indicator for the influence of order-disorder transitions caused by changes in internal and/or external parameters, for example, by cosmetic treatment of the hair. That is, from the endothermic peaks recorded in the thermogram of human hair, statements about strength or damage to the human hair fiber can be made on the basis of peak position (transformation point) and peak area (transformation enthalpy).

Extensive studies on the influence of cystine content on the denaturation of α-helices in keratins have shown, for example, that the denaturation temperature (transition temperature) of the keratin increases linearly with the cystine content. The increased stability of the matrix region due to the higher degree of crosslinking of the increased proportion of disulfide bonds in the matrix leads to difficulty in transforming the helices embedded in this matrix and thus results in an increase in the denaturation temperature. Conversely, a denaturation temperature and, above all, denaturation enthalpy decrease in human hair treated by perming or bleaching or dyeing can generally be observed.

2.2. Execution

Human hair strands (Kerling International (Backnang, Germany) “European natural hair 7/0”) were first bleached.

The two oxidizing agent preparations (O×1) and (O×2) were prepared for the bleaching.

Oxidizing agent preparation

| | |
|---|---|
| | OX 1 |
| Britesil C 265 (sodium silicate, SiO₂ content: 57.0-59.9% by | 36.0 |
| weight, Na₂O content: 21.3-22.8% by weight, molar ratio of | |
| SiO₂/Na₂O = 2.65 | |
| Magnesium carbonate | 9.65 |
| Sodium hexametaphosphate | 0.20 |
| Rohagit S hv (methyl methacrylate, methacrylic acid copolymer | 1.00 |
| (Evonik) | |
| EDTA | 2.00 |
| Silica | 0.4 |
| Glycine | 0.6 |
| Cekol 50000 (carboxymethyl cellulose, sodium salt (cellulose | 2.0 |
| gum) (CP Kelco, Nordmann Rassmann) | |
| Potassium persulfate | 32.00 |
| Ammonium persulfate | 10.00 |
| Sodium persulfate | — |
| Ariabel Blue 300302 | 0.15 |
| Dimethicone, dimethiconol | 1.50 |
| Paraffin Liquidum | ad 100 |
| | OX2 |
| Emulgade F (Cetearyl Alcohol, PEG-40 Castor Oil, Sodium | 4.0 |
| Cetearyl Sulfate (BASF) | |
| Potassium hydroxide | 0.1 |
| Sodium benzoate | 0.1 |
| Disodium pyrophosphate | 0.1 |
| Dipicolinic acid | 0.1 |
| Etidronic acid (60% aqueous solution) | 0.3 |
| Paraffin Liquidum | 17.0 |
| Hydrogen peroxide (50% aqueous solution) | 18.2 |
| Water | ad 100 |

The formulations (O×1) and (O×2) were mixed together in a weight ratio of 1:2 for the bleaching. This mixture of OX1 and OX2 represents the ready-to-use bleaching agent. The ready-to-use bleaching agent was applied to hair strands and left there for 45 minutes at 35° C. in a drying cabinet. After that, the hair strands were first rinsed with a shampoo, then with water and then dried.

Subsequently, the bleached strands were each dipped for 5 minutes in the formulations V (comparison), E1 and E2. After that, excess amounts of V, E1 and E2 were mechanically stripped off the strands. Subsequently, the strands were dried for 30 minutes with the hair dryer.

Then a straightening iron (brand: Schwarzkopf Professional; Model no: IP30A) was heated at the highest level for at least 10 minutes. The temperature measured with a contact thermometer (brand: Qtemp 300) between the heating plates was 230° C. after 10 minutes.

Now the dried hair strand was smoothed with the preheated straightening iron. Attention was paid to the following guidance of the straightening iron along the hair fiber: 4 times short (1 second per hair strand), 2 times long (5 seconds per hair strand).

The above-described smoothing with the straightening iron was repeated three times per hair strand.

By use of thermoanalytical examinations (DSC device: Perkin Elmer DSC 7, 60 μl stainless steel crucible with O-ring (24 atm)) test compositions were then tested for structural stabilization after the repeated heat application. Each strand was divided into parts of approx. 1 mm each. 12 of these aliquots of the same strand were filled in a DSC measuring crucible and 50 μl bidistilled water added. The measuring crucibles were closed and the sample measured in a temperature range of 100° C. to 170° C. (heating rate: 10 Kelvin per minute).

5 strands were tested for each test composition. The keratin melting points calculated from the individual measurements by arithmetic mean are listed in Table 2. The lower the keratin melting point, the more the hair strand is damaged.

TABLE 2
Damage to the hair structure

| | Keratin melting |
|---|---|
| Treatment | point [° C.] |
| untreated original hair | 153.1 |
| bleached hair | 145.1 |
| bleached hair, treatment with agent (V), | 143.2 |
| 3 x smoothed | |
| bleached hair, treatment with agent (E1), | 151.7 |
| 3 x smoothed | |
| bleached hair, treatment with agent (E2), | 150.3 |
| 3 x smoothed | |

The keratin melting point of strands treated with E1 and with E2 is significantly higher than the fibers treated with the non-inventive test composition V.

In addition, the keratin melting point of the stands treated with E1 and with E2 is even higher than the keratin melting point of the bleached hair.

The hair damage caused by the oxidative hair treatment could be effectively minimized by using the agents (E1) and (E2) and the subsequent heat treatment.

### 3. Formulation Examples

Conditioner

| | % by weight |
|---|---|
| Cetearyl alcohol | 4.0 |
| Ceteareth-20 | 0.3 |
| Isopropyl myristate | 0.8 |
| Glyceryl stearate | 0.3 |
| Stearamidopropyl dimethylamine | 0.4 |
| Varisoft W 575 PG (QUATERNIUM-87, | 4.0 |
| PROPYLENE GLYCOL), Evonik | |
| Dehyquart F 75 T (DISTEAROYLETHYL | 1.0 |
| HYDROXYETHYLMONIUM METHOSULFATE, | |
| CETEARYL ALCOHOL) BASF | |
| Methylparaben (Na salt) | 0.3 |
| Cosmedia Triple C (Polyquatemium-37, | 0.3 |
| Dicaprylyl Carbonate, | |
| Lauryl Glucoside) BASF | |
| Citric acid | 0.45 |
| D-panthenol | 0.2 |
| Phenoxyethanol | 0.4 |
| Nutrilan Keratin W PP (Hydrolyzed | |
| Keratin) BASF | |
| Ethylene carbonate | 5.0 |
| 1,2-propanediol | 10.0 |
| Water | ad 100 |

Cure Strengthener

| | % by weight |
|---|---|
| Ethanol | 35.0 |
| PVP/VA copolymer (30/70) | 8.0 |
| PVP/VA copolymer (60/40) | 8.0 |
| Cetrimonium chloride | 1.0 |
| D-panthenol | 0.25 |
| Lactic acid (85% in water) | 0.02 |
| Ethylene carbonate | 5.0 |
| 1,2-propanediol | 10.0 |
| Water | ad 100 |

Foam

| | % by weight |
|---|---|
| Cocoamidopropylbetaine (40% in water) | 5.0 |
| D-panthenol | 0.3 |
| Glycerol | 0.4 |
| PEG-40 Hydrogenated Castor Oil | 0.4 |
| Ethylene carbonate | 5.0 |
| 1,2-propanediol | 10.0 |
| Water | 69.0 |
| Propane/butane | ad 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An agent (M) for the treatment of keratin fibers, comprising in a cosmetic carrier, based on the total weight of the agent (M),
(a) one or more aliphatic polyols in a total amount from about 1.0 to about 40.0% by weight and
(b) one or more cyclic carbonates in a total amount from about 1.0 to about 20.0% by weight,
wherein the weight ratio of the total amount of the aliphatic polyols (a) included in the agent (M) to the total amount of the cyclic carbonates (b) included in the agent (M), (the weight ratio (a)/(b)), is a value of from about 40 to about 1.

2. The agent according to claim 1, wherein the
(a) one or more aliphatic polyols (a) is selected from the group of 1,2-propanediol, 1,2-ethanediol, 1,2,3-propanetriol, 1,3-propanediol, 1,2-butanediol, 1,3-butanediol and 1,4-butanediol.

3. The agent according to claim 1, wherein the
(a) one or more aliphatic polyols is present in a total amount from about 5.0 to about 20.0% by weight.

4. The agent according to claim 1, wherein the
(b) one or more cyclic carbonates is selected from the group of ethylene carbonate, propylene carbonate, butylene carbonate and glycerol carbonate.

5. The agent according to claim 1, wherein the
(b) one or more cyclic carbonates is present in a total amount from about 2.0 to about 15.0% by weight.

6. The agent according to claim 1, wherein the weight ratio of the total amount of the aliphatic polyols (a) included in the agent (M) to the total amount of cyclic carbonates (b) included in the agent (M), (the weight ratio (a)/(b)), is a value of from about 10 to about 1.

7. A method of treating hair comprising the following steps in the order specified:
(I) treating the hair with an agent (M),
(II) letting the agent (M) act on the hair,
(III) optionally rinsing off the agent (M) from the hair,
(IV) optionally drying the hair and
(V) heat treatment of the hair,
wherein the agent (M) is according to claim 1.

8. The method according to claim 7, comprising
(I) treating bleached and/or lightened hair with the agent (M).

9. The method according to claim 7, comprising the following steps in the order specified
(I) treating the hair with an agent (M),
(II) letting the agent (M) act on the hair for a period of from about 2 to about 45 minutes,
(IV) drying the hair and
(V) heat treatment of the hair,
wherein the method is free of the step (III) of rinsing off the agent (M) from the hair.

10. The method according to claim 7, wherein the hair in step (V) is subjected to a heat treatment at a temperature from about 50° C. to about 350° C.

11. The method according to claim 7, wherein the heat source for the heat treatment in step (V) is a straightening iron or waving iron.

12. A multi-component packaging unit (kit-of-parts), comprising at least two cosmetic agents (M) and (OX) assembled separately from each other, wherein
the agent (M) is according to claim 1; and
the agent (OX) is an oxidizing agent preparation comprising at least one oxidizing agent selected from the group of hydrogen peroxide, ammonium persulfate, sodium persulfate and potassium persulfate.

13. (canceled)

14. The agent according to claim 2 wherein the agent (M), based on its total weight, comprises (a) one or more aliphatic polyols in a total amount from about 5.0 to about 20.0% by weight.

15. The agent according to claim 1 wherein the agent (M), based on its total weight, comprises (a) one or more aliphatic polyols in a total amount from about 8.0 to about 18.0% by weight.

16. The agent according to claim 1 wherein the agent (M), based on its total weight, comprises (a) one or more aliphatic polyols in a total amount from about 9.0 to about 16.0% by weight.

17. The agent according to claim 1 wherein the agent (M), based on its total weight, comprises (a) one or more aliphatic polyols in a total amount from about 11.0 to about 16.0% by weight.

18. The agent according to claim 2 wherein the (b) one or more cyclic carbonates are selected from the group of ethylene carbonate, propylene carbonate, butylene carbonate and glycerol carbonate.

19. The agent according to claim 2 wherein the (b) one or more cyclic carbonates are selected from the group of ethylene carbonate and propylene carbonate.

20. The agent according to claim 1 wherein the agent (M), based on its total weight, comprises (b) one or more cyclic carbonates in a total amount from about 4.0 to about 9.0% by weight.

21. The agent according to claim 1,
wherein the (a) one or more aliphatic polyols (a) is selected from the group of 1,2-propanediol and 1,2-ethanediol,
wherein the (a) one or more aliphatic polyols is present in a total amount from about 11.0 to about 16.0% by weight,
wherein the (b) one or more cyclic carbonates is selected from the group of ethylene carbonate and propylene carbonate,
wherein the (b) one or more cyclic carbonates is present in a total amount from about 4.0 to about 9.0% by weight, and
wherein the weight ratio of the total amount of the aliphatic polyols (a) included in the agent (M) to the total amount of cyclic carbonates (b) included in the agent (M), is at a value of from about 3 to about 2.
